(19) United States
(12) Patent Application Publication
(10) Pub. No.: US 2024/0148297 A1
(43) Pub. Date: May 9, 2024

(54) PTSD RECONSOLIDATION WITH USE OF CALIBRATION AND BREAK STATE TOOLS

(71) Applicant: Frank J. Bourke, Corning, NY (US)

(72) Inventor: Frank J. Bourke, Corning, NY (US)

(21) Appl. No.: 18/413,811

(22) Filed: Jan. 16, 2024

Related U.S. Application Data
(63) Continuation-in-part of application No. 17/728,685, filed on Apr. 25, 2022.
Application No. 17/728,685 is a continuation-in-part of application No. 15/142,438, filed on Apr. 29, 2016, now abandoned.
Application No. 15/142,438 is a continuation-in-part of application No. 15/016,627, filed on Feb. 5, 2016, now abandoned.
Application No. 15/016,627 is a continuation-in-part of application No. 13/770,556, filed on Feb. 19, 2013, now abandoned.
(60) Provisional application No. 61/600,174, filed on Feb. 17, 2012.

(51) Int. Cl. A61B 5/16; A61B 5/0205

(52) U.S. Cl. CPC A61B 5/165; A61B 5/0205; A61B 5/1116; A61B 5/163

(57) ABSTRACT

A method supported by tools for determining specific steps to modify the traumatic memory of a PTSD patient. Specifically, the tools monitor four mental states indicating memory activation and others using measurable and observable physiological factors and behaviors.

### CROSS-REFERENCE TO RELATED APPLICATIONS

This application is a Continuation-in-part of U.S. patent application Ser. No. 17/728,685, which is Continuation-in-part of U.S. patent application Ser. No. 15/142,438 filed Apr. 29, 2016, which is a Continuation-in-part of U.S. patent application Ser. No. 15/016,627 filed Feb. 5, 2016, which is a continuation-in-part of U.S. patent application Ser. No. 13/770,556 filed Feb. 19, 2013, which claims priority to U.S. Provisional Patent Application Ser. No. 61/600,174 filed Feb. 17, 2012, the entirety of which is incorporated herein by reference.

### BACKGROUND OF THE INVENTION

The invention is a neurological intervention for the treatment of PTSD and other trauma reactions.

PTSD is a debilitating mental illness that impacts the lives of countless Veterans, active combat service members, Veterans, first responders, crime victims and others who have been exposed to life threatening or horrifying circumstances.

All of the front-line behavioral interventions, despite having been accorded the status of evidence-based treatments, fall far short of expectations. Consistent research has shown that all of these behavioral interventions are equivalent in results (Steenkamp & Litz, 2013; Steenkamp, Litz, Hoge, & Marmar, 2015) and the results, while better than nothing are not impressive (Bisson & Andrew, 2007; Goodson et al. 2011; Steenkamp & Litz, 2014).

Of the two treatments reviewed by Steenkamp and associates (2015), both obtained clinically significant reductions in symptom ratings (10-12 points; Monson, Gradus, et al., 2008; Schnurr, Friedman, et al., 2003) for both self-report and physician administered measures. Both interventions tended to lose symptom reductions at follow-up. Loss of diagnosis, when reported ranged from 17% to 39% of cases.

A 2013 Cochrane systematic review of PTSD treatments for adults (Bisson, Roberts, Andrew, Cooper, & Lewis, 2013) examined 70 studies involving a total of 4761 individual participants. Their analysis found that trauma-focused cognitive behavioral therapies (TFCBT), including PE and CPT, were better than waitlist and treatment as usual controls. The mean severity of symptoms typically dropped by 1.6 s standard deviations.

Standard behavioral treatments for PTSD are largely dependent upon extinction protocols that make use of various exposure-based methods. Such treatments have several drawbacks. All or nearly all require adjunctive or follow-up treatment (Foa, et al., 2000). All risk retraumatization of the patient and minimally require some significant level of re-exposure to the traumatizing event. Insofar as they are extinction-based, positive treatment results tend to fade over time as the hallmarks of the extinction process spontaneous recovery, contextual renewal, reinstatement, and rapid reacquisition assert themselves (Bouton, 2004; Rescorla, 1988; Schiller, Kanen, LeDoux, Monfils, & Phelps, 2013)).

The invention described herein departs from previous art in that it is rooted in a neurological mechanism known as reconsolidation blockade and relies upon empirical observation and monitoring, not abstract theory. The claimed protocol uses data based inputs to determine neurological states of the patient's memory and structure threreof with the result of a high probability of remission of the and loss of diagnosis.

An unpublished pilot study of the RTM intervention was completed in San Diego, CA. That study, a 30 person wait list controlled protocol with blinded psychological measures, replicated the findings of the earlier study.

An earlier search of the journal literature for memory mechanisms that explain the pattern observed with the protocol found that the reconsolidation mechanism was the only pattern of application and efficacy that matched the reported results. Although reconsolidation has been observed in all memory systems, the following information is focused upon its application to emotional memones (Gray & Bourke, 2015).

Reconsolidation describes the reactivation of long term, otherwise permanent memories, by their evocation in certain contexts. When a memory is reactivated, it becomes subject to change. If the circumstances surrounding the memory remain the same, the memory remains unchanged; it reconsolidated and is maintained in its current state. If circumstances have intensified, the impact of the memory may become worse; re-traumatization can add to the intensity of trauma memories; the reconsolidation process allows complimentary information to be added to the memory. If the new circumstance provides evidence that the threat predicted by the trigging stimulus (CS) is no longer relevant, the strength of the affective charge may decrease; the new information blockades the emotional response to the memory and adds in the new information. New information, relevant to the original memory, especially when novel, supports reconsolidation. Without new, relevant information, memory updating through the reconsolidation mechanism does not occur. New information produces a so-called prediction error. A prediction error is a presentation that violates expectations about the circumstance predicted by the stimulus. This may be created by not requiring a complete retelling of the trauma, telling it in a new circumstance, voluntarily telling the story, and/or manipulating the structure of the experience. The introduction of new information blockades the reassertion of the old memory structure and transforms it.

The procedure starts with a brief, controlled reminder of the trauma that, according to reconsolidation theory, renders the traumatic memory subject to change. The reminder of the traumatic memory is interrupted and never allowed to bloom into a full, traumatic, re-experiencing event using a calibration tool that warns of and predicts retraumatization episodes. The calibration tool also allows for determination that the trauma memory has been activated. After a period of 10 minutes, the old memory enters a period of malleability (lability) in which it becomes subject to change. This so-called ‘window of lability’ may last as long as six hours. The procedure then adds in dissociative experiences which transforms the affective response to the memory. After treatment, the memory is accessible but non-traumatizing. Unlike extinction-based protocols, the disturbing symptoms are not subject to spontaneous recovery, contextual renewal and other extinction phenomena; the symptoms disappear and stay away. After treatment, the traumatic event becomes available to non-traumatizing declarative access for patient typically and spontaneously as a distant, non-threatening memory.

It is the object of the present invention to provide a new, brief, clinician-guided, non-traumatizing intervention for the elimination of PTSD characterized by intrusive symptoms. The intrusive symptoms of PTSD that provides a kit of tools to the clinician to guide the clinician to apply the various aspects of treatment in appropriate and effective order. It is estimated that 50 to 75% of all PTSD patients suffer from this variety of the disorder.

The invention is distinct from therapies aimed at the disorder in that it is a neurological intervention, rooted in the syntax of memory reconsolidation blockade and deals directly with the neurological structure of the remembered trauma. The invention is a physical method of activating the neurological memory structure and applying direction determined by observable factors. While the visual and dissociative formats employed by the intervention may be altered (as detailed in TABLE N), the reconsolidation format underlying the visual and dissociative specifics is critical to their formulation and the protocol's efficacy but must be guided by the use of tools.

The mechanism upon which the invention relies is distinct from extinction, the neurological process upon which PTSD therapies generally judged to be most effective are based in that it is not subject to spontaneous recovery, contextual renewal and the other hallmarks of extinction learning.

The invention is distinct from other interventions for PTSD in that it adheres closely to the pre-clinical syntax of reconsolidation blockade including: 1) identification of a target, previously existing, trauma memory; 2) a brief non-reinforced or interrupted reminder of that target memory; 3) a pause or distraction; 4) the introduction of new, relevant, or novel information conceived as changes in the structure of the formatting of the visual, kinesthetic and auditory elements of the trauma memory; 5) following the intervention the memory is retested to assess its transformation or lack thereof.

The invention is also distinct from other PTSD interventions in that patients who complete the intervention typically and spontaneously reappraise the meaning of the memory, find an appropriate context for the memory, gain new perspective about the memory, and reintegrate the traumatic memory, all without specific guidance from the clinician.

The invention requires the guidance of a clinician who applies specific tools to determine the activations of the index memory and the level of stress being experienced by the patient because there is potential for adverse emotional reactions and the process is generally too complex to be managed without guidance. Using described tools, the clinician is needed in order to: 1) guide the patient through the intervention process; 2) apply the use of tools that assess autonomic reactivity at various points in the intervention; and 3) provide a safe means of freeing the patient from overwhelming responses should they occur.

The clinician also using a calibration tool determines the extent of the patients ability to adhere to and their adherence to instructions given by the clinician. By use of instructions to 1) allow the patient to safely and directly experience the memory, 2) terminate the expression of the memory to prevent panic reactions so that the intervention can continue, and 30 temper the immediacy of the memory through the use of structural changes to the memory. These instructions determine when the patient index memory is activated, and how it will be handled by use of directions to disassociate the patient from experiencing the memory directly rather than through abstractions that temper the immediacy of the memory to evoke panic reactions. What is described herein is an approach to direct the patient to imagine the memory from a distance, in third person, from an out of body perspective, in settings that are safe and create a ready availability to modify aspects of the memory. This abstraction process also allows the clinician to explore the patient's memory while the calibration tool monitors the patient's mental state indicating index trauma memory activation and lability. In the event the calibration tool gives rise to concerns about high stress levels that could lead to re-traumatization the clinician can use break state tools which are a set of verbal and non verbal cues that lead the patient into a disassociated state from the index trauma memory. The main phases and components of the memory are:
1) The patient identifies a most important or most significant index trauma related to current experiences of nightmares, flashbacks and intrusive thoughts. This is typically the one most closely related to the content of the flashbacks and nightmares that are the central symptoms for the patient. In cases where there is a series of events or the index trauma extends over a long time period, the patient is encouraged to identify the single, central, or most important incident.
2) The intervention may be continued either with full or partial disclosure of the content or as a content free intervention.
3) The clinician asks the patient to relate the trauma narrative as fully as possible, or to imagine encountering the kind of event that triggers a typical flashback. In many cases the patient will be so overcome with emotion and automatic negative responses that they cannot relate the entire story.
4) The clinician must observe the patient closely and as soon as the patient begins to show signs of autonomic arousal (while relating the event), he is to stop the retelling and reorient the patient to the present time and context. These signs of autonomic arousal are measured by a calibration tool that tracks factors such as physiological factors, behaviors, and measurable autonomic reactions.
5) The clinician then asks the patient to describe two neutral events, one from just before the traumatic event (neutral start) and a second from after the event, when the patient knew that event itself was over, and they had survived (neutral end). The patient is then asked to imagine entering a movie theater where the neutral start picture is on the screen in black and white.
6) After orienting to the theater, choosing a seat, and sitting down, the patient is to imagine floating up out of their body and into the projection booth from which they can see their body in the theater and the screen displaying the B&W neutral beginning picture.
7) At the clinician's instruction, the patient is to watch their own body in the theater as that body watchesa black and white movie of the trauma event on the movie screen. The movie begins with the neutral start image and ends with the neutral end image. After running the movie, the patient is asked to rate their comfortability with the movie process. The movie process with a neutral memory can be practiced to allow the clinician to determine whether the patient understands the instructions and follows directions, and to facilitate their capacity to visualize the movie.
8) If there was discomfort the black and white movie is rerun with modifications as needed (as described in the DETAILED DESCRIPTION, Table 5 below) and as many times as needed until the B&W movie produces no discomfort.
9) When the movie scenario feels comfortable, patient is invited (on cue) to float back down into their own body and walk to the movie screen where they will step into the neutral end picture (still on the screen), restore color and sound to the image and, in about two seconds, relive the entire scenario fully associated, in reverse, experiencing the event undoing itself from within, ending at the safe, neutral beginning picture (Neutral start). This may be repeated with adjustments (as described in DETAILED DESCRIPTION below) until it can be completed comfortably.
10) The patient is now asked to relate the trauma narrative. When the narrative can be repeated with little or no impact, as if at a distance with increased detail, the process continues to the next phase. If there is still strong autonomic responsivity, the entire movie sequence may be repeated.
11) The patient is now invited to design a new version of the trauma scenario with increased and fluidity in which either they were not injured, different choices were made, the event did not occur, or that others were not injured.
12) When a satisfactory exemplar has been constructed, the patient imagines going through it in the version of the trauma scenario, beginning at the neutral start, in which either first position.
13) If a first run through of the revised scenario is not acceptable, a new scenario may be generated. When a satisfactory scenario has been identified, it may be repeated six to eight times.
14) The patient is now asked to relate the trauma narrative. When the trauma narrative can be repeated without discomfort, as if at a distance with increased detail and fluidity, the process is presumed to be over. If there is still strong autonomic responsivity, the rescripted scenario, or a new rescripted scenario may be repeated with a different set of modifications. The discomfort is measured by the calibration tool taking into account all of the measured and measurable factors.

While this specific visual/dissociative sequence has been developed as a highly effective generalization, successful administration sometimes necessitates alteration of the specific components while adhering to the underlying reconsolidation format. These and further details and alternative presentations are presented in the detailed description below.

### DETAILED DESCRIPTION

Inclusion and Exclusion Criteria

The method described defines specific inclusion and exclusion criteria:

The patient must meet criteria for a diagnosis of PTSD by a standard DSM or ICD-11 criterion via methods described below. It is essential that the patient's difficulties are essentially a phobic, instantaneous conditioned response to the narrative of a traumatic event that threatened immanent death or injury to self or others, an element of that narrative, or a stimulus related to the involuntary evocation of the aforesaid traumatic event.

Where multiple traumas are involved each must be separable from the others as a distinct event. That is: each event must have an identifiable starting and end point even if they belonged to the same broad emotional, geographic or temporal context.

The problem must 1) Be rooted in the personal experience of trauma threatening death or injury to oneself or others, and 2) Be expressed as an intense, suddenly arising experience of the trauma symptoms usually experienced as flashbacks or panic reactions.

Additionally, the central symptoms for the appropriate use of the protocol are recurrent flashbacks (at least once a month) and nightmares (at least once a month). If these intrusive elements are missing, the protocol may be inappropriate.

Where such nightmares and or flashbacks are present, the intervention is appropriate when, in the course of describing the index trauma, or when exposed to a triggering stimulus, the patient displays signs of autonomic arousal which may include but are not limited to, freezing, flushing, shaking, sweating, changes in facial color, involuntary motor responses, changes in breath rate, etc. In addition, while relating a trauma narrative, such arousal may be reflected by pauses, loss of detail, loss of fluidity, inability to speak, pressured speech, etc. (see Table 1, below).

The traumatic response must be in regard of a specific event; it may not be a response to the meanings of the event in the patient's larger life or the impact of such events on the patient's sense of self-worth. Such excluded problems may include, but are not limited to, grief, guilt, shame, anxiety, and anger.

If both kinds of response are present, the patient must be advised that the protocol will work for the intrusive elements with an immediately arising phobic-like response. RTM will eliminate flashbacks and other immediate panic responses to reminders of the traumatic event—but may have no effect on larger life issues which will require other kinds of treatment.

While the invention targets the intrusive symptoms of PTSD, its use may be associated with the ending of other co-occurring problems which are secondary responses to the nightmares and flashbacks themselves. Such relationships have been observed with substance use disorders, hypervigilant, and avoidant symptoms; nevertheless, these will not be affected in all cases.

Rapport

The therapeutic perspective adopted in the RTM process assumes that change work occurs in the subjective space between two or more people. It also assumes that each person has a distinctive representation of the world that does not in all points correspond either to another person's representation of reality or to the common reality with which they must deal. In order to communicate across this representational gap and to establish a common set of understandings for such communication, it is important for the clinician to understand and at least provisionally accept the patients understanding of the world and of their current problem. This acceptance is facilitated by the understanding that the method here described deals with the subjective structure of the problem, not its content. Rapport is an essential tool in this regard.

Rapport may be understood as the establishment of a state of trust or empathy between persons. It may also be understood as a state of mutual sensitivity to the meanings and values held by the participants in the communication.

Rapport may be established by matching the patient's use of language predicates and their paralinguistic behaviors. Specific techniques may include matching posture, breathing, predicates, voice tone, rate and volume, etc. In general, however, presenting an open and honest concern for the patient's outcome, and carefully eliciting feedback to ensure that you have correctly understood their meaning usually produces the most significant level of rapport. Rapport is usually manifested by a dance of matched movement, breathing and other paralinguistic elements as the participants consciously or unconsciously exchange nonverbal signals (Bandler & Grinder, 1979, Gray, 2011). In this context, rapport does not guarantee self-disclosure, only cooperation with the treatment.

Rapport may also be crucial for trauma work as it can provide a significant link to the present as a safe place or context in case the elicitation of the problem state becomes too intense. Insofar as a strong rapport has been established, it serves as a positive physiological reference point for the RTM process itself. The establishment of rapport may be considered a conditioned response to a positive present state elicited by the clinician's physical presence. In such cases the patient can be called back into the present moment by commenting on something, the request that they look the clinician in the eye and breathe with them, or some other reminder of the present.

Framing the Invention

Framing provides a meaningful context for the intervention. It sets up specific expectations about the procedure, gains crucial permissions and is used to defuse any anxiety associated with treatment. In practice, it allows the clinician to set the limits on discomfort that the patient may experience and serves to review the general procedures to be used. Framing also serves to separate the RTM intervention from previous forms of treatment that may have been unpleasant or ineffective. It does so by discussing the distinctive features of the invention as contrasted with treatments that are ineffective or employ techniques that include the possibility of retraumatization through exposure to the index trauma in vivo, in silico, or in imago. In doing so, it serves to eliminate anxieties related to reliving the trauma or losing control. Finally, the frame is important in establishing a relationship in which the patient is empowered to stop and question the procedure as necessary.

The Framing Procedure:

Explain that you will be doing a short visualization process that is ordinarily comfortable, but sometimes has a very short period of moderate discomfort.

Assure the patient that at the first signs of discomfort, you will stop the procedure and make adjustments as necessary.

Ask about any previous therapy or attempted interventions and explain how the process you will use is very different, in particular, explain that RTM does not involve “reliving” the traumatic events through exposure or “catharsis” or “release” of feelings.

Then ask, “Do you have any questions or concerns before we begin?” If the patient has any concerns about doing this process, respond to them congruently, and assure them that if any questions or concerns arise at any time during the process, it is fine to interrupt it and to tell you what they are.

As part of the framing process, before the active intervention begins, have the patient practice the central parts of the intervention with one or two neutral innocuous experiences.

There are at least two parts of the intervention that are sufficiently complex that they often require that the patient practice them before proceeding. This complexity and the need for providing guidance to the patient is one of the reasons that a trained clinician is essential to the process.

The two elements that typically require practice are the black and white dissociated movie and the fully associated rewind.

Practice

Explain to the patient that she will be doing some things in her imagination that she may never have done before and that it would be useful to have some practice. After obtaining her consent, ask the patient to think of a neutral or mildly negative experience. Have them identify a place or time just before the identified scene clearly begins (neutral start) and a place or time where the scene has clearly ended (neutral end). For example, for a scene representing a memory of eating lunch, the start image might be walking through the doorway into the kitchen. The end scene might represent putting the dishes in the sink. Each scene clearly marks a point outside of the event described as ‘eating lunch.’ The neutral start and end images are to be held as still, black and white images that signal the boundary of the target memory. Have the patient practice viewing the experience from beginning to end as a black and white movie projected on a wall or an imagined screen outside of themselves. Each iteration of the practice movie begins with the still, neutral start image and ends with the still neutral end image. Do this until the patient can perform the task with ease. Similarly, after stopping the black and white movie, have the patient practice stepping into the end frame of the black and white movie (neutral end), turning on the colors and sounds and running the entire scenario backwards as an imaginal, present time, fully associated experience of the same movie, lasting only about two seconds. Have the patient practice these two elements one-at-a-time until they are done easily and then proceed with the treatment.

Elicitation of the Traumatic Response

According to Foa and colleagues (Foa, Keane, & Friedman, 2000; Foa and Kozak, 1986; Foa & Meadows, 1997), elicitation of the problem state is the sine qua non of effective trauma treatment. They also warn specifically against the use of ineffective probes or questioning techniques that fail to elicit a valid physiological experience of the trauma response. The RTM protocol relies on multiple steps, and the assessment of physiological concomitants of the patient's response at each step. A simple verbal answer regarding the problem state is never sufficient; the clinician must be able to identify the specific signals of the fear response as they arise in the present time (See Table 1). If there is no physiological response indicating that the phobic response has been triggered, the intervention will not be successful. Likewise, at the end of the protocol it is important to verify that the fear response is no longer generated in response to the previous triggers.

Although the preferred method for using the intervention is to elicit some kind of description of the traumatic incident, this intervention may be pursued content free. That is, the clinician does not need to know the specific content of the presenting problem, and the questions don't need to be answered verbally; they are only asked in order to elicit the physiological changes, and to verify that these are immediate and intense. However, brief content information about triggers can be useful at the end of the protocol to verify that the patient has a neutral response to them.

Trauma Narrative

Accordingly, after establishing rapport, the clinician should ask about the target event state in order to elicit the physiological response. The following kind of question is suggested, allowing only enough of a response to elicit the problem response: What is the worst time that you had your problem response? (flashback, panic, etc.) When and where does this typically occur? (crowded rooms, open spaces, etc.) What triggers your response? (Humvees, loud noises, Arab clothing, etc.) More simply, the clinician may ask: “Tell me what happened?” As the patient responds, the clinician must attend to the physiological and paralinguistic elements that reflect heightened arousal and the elicitation of the problem response. One of the most reliable signs of having identified an appropriate trigger is the fast onset of the physiological and paralinguistic symptoms of fear or trauma. Table 1 lists these symptoms.

TABLE 1
Signs of Autonomic responsivity comprising:

Changes in breath rate
Changes in heart rate
Changes in muscular tension
Changes in skin color
Changes in skin tone
Changes in voice pitch
Changes in speech rate
Flushing
Freezing
Involuntary motor responses
Loss of detail in narrative
Loss of fluidity in narrative
Mutism
Pauses in narrative
Postural changes
Pressured speech
Shaking
Sweating
Tremors

It may be useful to make notes as to what the patient was saying as the symptoms began, whether they were focused inwardly or outwardly, or any other changes observed.

A response to these probes, by an appropriate patient, usually results in a narrative that is truncated, lacking details, characterized by short vignettes punctuated by strong autonomic arousal. It may be impossible for the patient to complete the narrative.

These responses serve several essential purposes. 1. They allow the clinician to clearly identify the problem response in terms of its physiological expression and to verify that it is an appropriate phobic response that arises quickly and intensely. 2. They allow the clinician to identify the specific trigger or stimulus that evokes the state in terms of the patient's own descriptive behavior at the time of symptom onset. 3. By providing a typical response to the eliciting stimulus, they provide information that can be used to identify the problem state if it re-emerges in the testing phase after a failed or incomplete treatment. 4. The absence of this response to triggers in the testing phase will signal the success of the treatment. 5. According to reconsolidation theory, the process cannot begin until a short reminder stimulus (enough to awaken autonomic responsivity) has been elicited.

Eliciting the nonverbal problem response engages the clinician and patient in a reciprocal relationship that arouses the autonomic system, sensitizing the patient to the relevant stimuli. The active participation of the patient in consciously accessing the problem state begins the cognitive restructuring of the problem state using the technique of prescribing the symptom. The deliberate elicitation of the flashback or phobic state represents the reminder phase of the reconsolidation process and represents a novel context that facilitates the labilization of the trauma memory. By actively participating in eliciting the problem state, the patient gains an experience of control over what was previously an uncontrollable and unpredictable traumatic response. This is also an example of the therapeutic use of Bandura's concept of self-efficacy in the context of PTSD treatment.

The literature of Reconsolidation indicates that reconsolidation only occurs when the target memory is aroused in a novel context or a novel manner; if there is no new information, the memory will not become subject to updating.

From time to time, especially during the recitation of the trauma narrative and sometimes during other parts of the protocol, patients will become associated into a negative experience and begin to show signs of autonomic reactivity such that they are appear to moving towards a full flashback or abreaction. In such cases, it is essential, as soon as the first signs of arousal appear (Table 1) to stop the progress of the experience. The options used to break state are listed in Table 2 Breaking State. Other examples are available in the literature are listed in Table 2.

TABLE 2
Break State Patterns Comprising:

Distracting the client so as to return them to a neutral or resourceful state
by asking the client about their favorite music, favorite foods, where they
grew up, or any other innocuous topic
Orienting the client to a different sensory system (looking at a view of
nature, something in the room, listening to music or a sound)
Physically moving the client to a different position: e.g., walking around
the room
Picking up a conversation that ended before initiation of treatment as if
treatment never happened
Having the client become aware of their feet on the floor and their
presence in the room.
Looking the client in the eye while calling their name and instructing
them to be present

Abstraction Protocols for Exploration of Index Trauma and allowing Memory Structure Modification.

The Movie Theather Scenario

After each recitation of the narrative, part narrative, for internal access to the trauma memory, the patient provides an assessment of their response to the memory using the Subjective Units of Distress (SUD) score tool, as described below.

Help the patient to establish neutral start and neutral end points for the index trauma memory. Remind them that you are not going to delve into the memory but you just need to know what they were doing before it started and how they knew that the event itself was over using language comprising: What were you doing then? Find the neutral end point by asking (e.g.), “How did you know that this event was over? Just this moment, this event. What did you see and hear that let you know that it was done?”

Similarly, for the neutral start image (e.g.): “What were you doing just before this happened? Perhaps it was something completely different.”

Have the patient imagine the neutral start and neutral end images as still pictures that will bookmark the beginning and end of the black and white movie.

Ask the patient if they've ever been to a movie theater. Request that they recall a familiar one and recall how it was decorated, how it was arranged, the colors employed, how it smelled, etc.

Inform the patient that you will be using the movie theater as part of the intervention. In a few minutes, they will be watching themselves, watching themselves as they watch a black and white movie of the problem event. But that is not going to happen yet.

Ask the patient to imagine walking into that theater where there is, displayed on the screen, a black and white, still picture of the neutral start image. Have them describe the physical aspects of the theater comprising: whether there is a stage, a proscenium arch, curtains, the colors of the interior, the color of the chairs, the fabric of the chairs, whether the floor is carpeted or not?

Instruct the patient to walk half to three quarters of the way towards the rear of the theater to find a seat that they find comfortable. Ask them to describe the seat and its environs using questions comprising whether it folds or slides, whether it is adjustable or not, what the fabric is, whether it is comfortable, whether the floor is sticky or not, what the arm rests feel like, etc.

Once the patient is comfortably seated, remind them to notice the still black and white picture on the screen. Have them notice that it represents a still picture from the time before anything happened.

The Black and White Movie

Instruct the patient to listen to the following instructions first and then to complete them on cue.

Instruct the patient to imagine themselves floating up out of their own body, there in the theater, to a place in the projection booth, above and behind them. In the projection booth they are to walk up to and place their hands on a thick old, plexiglass window that looks out over the theater. Instruct them to appreciate the thickness of the window, to notice whether there is yellowing or scratches on it, and to look down into the theater to see their own body, down there, in the theater, looking at the black and white picture projected on the screen. Tell the patient to do this as you repeat the instructions. Have them relax in their chair and watch their own body, there in the theater, as it looks at the still picture.

Once the patient is situated in the projection booth, they should exhibit signs of dissociation comprising the elements listed in table 3.

TABLE 3
Signs of Dissociation comprising:

Client leans back in chair (they are not leaning forward; they are not
pointing or gesturing)
Head is back, chin is up
Posture is open (it is not folded in, or protective)
Facial expression is relaxed and symmetrical (there are no signs of
concentration or effort)
Muscles are relaxed (there is no obvious tension, they are not
holding on to themselves or the furniture)
Breathing is measured
Pulse is regular and even

The signs of Table 3 indicate a calibrated sign of disassociation and are checked to see whether they fit the model of disassociation of the particular patient as set in the calibration tool.

If the projection scenario does not provide an adequate level of dissociation as indicated by the elements comprising those listed in Table 3, other means may be used to enhance the patient's level of dissociation from the theater, his alternate position in the theater, and the still picture on the screen comprising those listed in Table 4.

TABLE 4
Methods Used to Enhance the Initial Dissociation in the Movie Theater:

From time to time it will become necessary to provide alternate means to assert an
appropriate level of dissociation in the client. This may be especially necessary during
the first part of the procedure in which the client may have difficulty dissociating from
his image in the theater, the neutral start image or the basic black and white move. The
methods, ordered by frequency of use and from the least intrusive to the most, comprise:

Moving the client from the projection booth to a ‘meta’, angelic, or divine
perspective above the theater and instructing them to watch themselves in the
projection booth as they watch the self in the theater as they watch the movie.
With and only with the client's explicit permission, gently pulling them back into
an upright position in the chair by gently tugging on their hair.
With and only with the client's permission, creating an anchor or conditioned
stimulus for the dissociative state by using a distinctive yet familiar comforting
touch to keep them mindful of a safe, dissociated state.
Similar modifications and distortions may be included to ensure dissociation
from the movie contents.

Once a satisfactory level of dissociation has been attained, instruct the patient to listen to the following instructions but not to execute them until given the cue.

On cue, but not before, the patient is to remain in the projection booth with their hands on the glass looking down at their alternate self in the theater (or other dissociative positions as suggested in Table 4), turn on the black and white movie, and watch themselves (down there in the theater), as they watch the black and white movie from beginning to end; starting with the neutral start image and ending with the neutral end image. At the end of the movie, the neutral end image is to remain on the movie screen.

It is important to emphasize to the patient that they are not to watch the movie itself. They are only to watch their alternate self, down there, in the theater, as they watch the movie. The patient should pay special attention on how the person, down there in the theater, responds. It may be useful to emphasize that there is no connection between the observer in the projection booth and the watcher in the theater. For all intents and purposes, they are unrelated.

When the patient is ready, have them start the movie, reminding them to focus upon the person in the theater and their responses, as the movie runs.

When the patient completes a run-through, whether or not there is evident autonomic arousal, the clinician should ask the patient, how the segment was for him and for the watcher in the theater. If either the patient (in the projection booth) or the watcher in the theater experienced any discomfort, the movie should be repeated or adjusted as follows. At all times during this process the autonomous responses of the patient are monitored to insure anxiety levels are not excessive and call for break state instructions.

During the running of the black and white movie, the clinician is to observe the patient for any of the signs of autonomic arousal as noted in Table 1. Mild arousal may be tolerable but at any point where the patient becomes clearly upset, or if the patient asks to stop, the clinician is to stop the procedure, break state using the methods described in table 2 (if necessary).

Once the patient is restored to the present context with no signs of continuing arousal, the clinician should negotiate, with the patient's full participation, an alternate presentation of the black and white movie using techniques comprising those listed in table 5.

TABLE 5
Methods Used to Enhance Dissociation:

From time to time it will become necessary to provide means to assert or reassert a level
of dissociation in the patient. This may be especially necessary during the first part of the
procedure in which the patient may have re-associated into the experience. of the black
and white move (described below). The methods, ordered by frequency of use and from
the least intrusive to the most, comprise:

| | |
|---|---|
| Moving the movie screen farther away | Moving the patient from the projection |
| from the watcher's seat in the theater | booth to a ‘meta’, angelic, or divine |
| Turning the movie screen or moving | perspective above the theater and |
| the watcher's seat (down in the | instructing them to watch themselves in the |
| theater) so that it is only visible at an | projection booth as they watch the self in |
| oblique angle. | the theater as they watch the movie. |
| Allowing the movie to be displayed as | With and only with the patient's explicit |
| first only the bottom half of the | permission, gently pulling them back into |
| screen, beginning to end, and then the | an upright position in the chair by gently |
| top part of the screen, beginning to | tugging on their hair. |
| end | With and only with the patient's permission, |
| Playing every odd numbered second | creating an anchor or conditioned stimulus |
| of the movie, beginning to end, | for the dissociative state by using a |
| followed by every even numbered | distinctive yet familiar comforting touch to |
| second of the movie beginning to end. | keep them mindful of a safe, dissociated |
| If there is sound (there is usually | state. |
| none), muffling, deadening or | Other similar modifications and distortions |
| distorting the sound so that it is no | may be included to ensure dissociation |
| longer bothersome. | from the movie contents. |
| Instead of using the projection booth | |
| scenario, having the client stand | |
| behind their own body in the theater, | |
| with their hands on their other selves' | |
| (the watcher's) shoulders, while | |
| attending to that other selves' | |
| response to the black and white movie | |

Having agreed on such a modification, the movie scenario is repeated (with more adjustments if needed) until the black and white movie can be completed with no discomfort. When the patient completes the black and white movie without incident it may be repeated several times before proceeding to the associated rewind. Each time that the movie is completed, at the end of the movie, the neutral end image is to remain on the movie screen.

Associated Reversal

Once the patient is comfortable with the black and white movie, instruct the patient to listen to the following instructions but not to execute them until given the cue.

The instruction to check for comfort in the body-in-the-theater before standing up and walking down. Float down from the projection booth and back into your body down there in the theater. After fully associating into your body, get up from your seat, walk to the isle and then walk down the aisle to the movie screen where the black and white, neutral end image is still displayed.

Go ahead and do that now.

Instruct the patient to listen to the following instructions but not to execute them until given the cue.

When I give you the instruction, step into the neutral end picture, turn on the colors and the sound, and the feeling, and experience the entire event moving backwards from the neutral end image to the neutral start image, all in about two seconds. Illustrate with a right to left gesture of the hand (if sitting beside the patient) while making a sound like, “Bezzouuuuurrrrppppp!”

Impress upon the patient that they are to experience the entire event as if they were experiencing it in reverse: everything is undoing itself; sounds run backward, motions are reversed, everything reverses its normal order. The scenario ends with the patient associated into the neutral start image.

When the patient indicates their understanding, tell them to step into the neutral end picture, turn on the colors, sounds and motions, and rewind the event.

During the running of the reversed associated movie, the clinician is to observe the patient for any of the signs of autonomic arousal as noted in Table 1. Mild arousal may be tolerable but at any point where the patient becomes clearly upset, or if the patient asks to stop, the clinician is to stop the procedure and break state using the methods described in Table 2 (if necessary).

Once the patient is restored to the present context with no signs of continuing arousal, the clinician should ask them about their experience, and identify any problematic parts of the reversed movie.

If there are significant problems with associating into the event at all, it may be necessary to redo the black and white movie scenario with new adjustments to ensure the patient's comfort. If there are mild problems, including brief pauses, or brief indicia of autonomic arousal it may be useful to repeat the reversal several times with specific instructions for seeing a motion (s) or action(s) in reverse, hearing a sound(s) in reverse, or other kinds of associated sensory reversals.

Once the patient is restored to the present context with no signs of continuing arousal, the clinician should ask them about their experience of the reversal. If no problems are reported the procedure continues with the narrative

Trauma Narrative

Ask the patient to tell you the story of the index trauma, beginning with the neutral start place, continuing through the event and ending with the neutral end scene.

If the narrative is completed without the signs of autonomies arousal detailed in Table 1, this segment of the intervention is deemed complete. If a significant level of arousal remains, the clinician must decide whether to repeat the entire procedure from the Movie theater scenario forward, or just the rewind.

When the narrative can be completed as a distant event, without autonomic arousal, or only minor arousal, proceed to the rescripting phase.

Rescripting

After recitation of the narrative or part narrative, the patient is to provide an SUD score for their experience of the trauma memory now.

If the SUD score is three or less, or three and trending lower, the process continues to the rescripting phase.

If the SUD score is three or more, or three and trending upwards, the process may revert to any earlier phase including the black and white movies and the associated rewind.

In all cases, the clinician's observations of the patient's physiology will be used to assess the veridicality of the SUD score.

After the patient has successfully retold the trauma narrative without significant arousal, he may proceed with the rescripted narrative.

Invite the patient to create a new or novel scenario for the trauma memory in which (despite beginning at the same neutral start): a) they were not injured; b) different choices were made that changed the nature of the incident; c) the event was a movie, play or charade of some kind; or d), the event did not occur. Allow the patient to design an alternate scenario that is acceptable to them.

Once an acceptable scenario has been designed, have the patient imagine going through it, first in a dissociated manner as if watching themselves from without. If this is acceptable, have the patient imaging experiencing the new movie from within fully present and associated: as if this was the way the event happened. Remind them that this is just a fantasy, and the truth remains the same, but that it will be useful to experience the event in this new way.

Have the patient run through the new version of the event once and check with them to ensure that it works for them. If the scenario needs changing, work with the patient to create a more acceptable version. Have the patient run through it once, and if satisfactory, have the patient repeat it four to eight times.

If the rescripted scenario can be repeated without problems, move to the narrative.

Narrative

Ask the patient to tell you the story of the index trauma, beginning with the neutral start place, continuing through the (actual) event and ending with the neutral end scene.

Immediately after recitation of the narrative, the clinician will obtain an SUD score from the patient.

If the SUD score is 1 or 0 and matches with the clinician's assessment using the calibration tools from Table 1 and the patient reports no distress, the treatment may be deemed complete.

If the SUD score is 2 or higher and it accords with the clinician's assessment of autonomic arousal using the calibration tools from table 1. The clinician may instruct the patient to repeat some previous facets of the protocol until the autonomic reactivity is satisfactorily resolved.

If the narrative is completed without the signs of autonomic arousal detailed in Table 1, the intervention is deemed complete. If a significant level of arousal remains, the clinician must decide whether to repeat the entire procedure from the Movie theater scenario forward, or just a new scenario.

When the narrative can be completed as a distant event, without autonomic arousal, full of detail, often accompanied by an ‘aha’ experience, the procedure is presumed to be over.

Specific Tools Used in the Protocol include the following tools: clinical assessment, diagnostic inventory tools; Subjective Units of Distress scores; Calibration; The Tool of Rapport; The Narrative Parsing Tool; The Tool of Structural Modification; The Break State Tool
1. The Tools of clinical assessment
2. The tool of diagnostic inventories allows the clinician to confirm (pre-existing) or establish a current diagnosis of PTSD and to establish the loss of diagnosis after completion of the RTM Protocol.
   a. Unlike therapies for PTSD, RTM is a neurological intervention that uses the inventories to assess remission, not simple reductions in symptom severity.
   b. RTM completely remits the diagnosis in most patients.
3. The tool of diagnostic inventories includes the PSS-I5, the PCL-5, the CAPS5, and other empirically validated instruments for the diagnosis of PTSD.
   a. Diagnosis of PTSD via the PSSI-5 requires a score≥23
   b. Remission via the PSSI-5 is defined as a score<23
   c. Diagnosis of PTSD via the PCL-5 requires a score≥33
   d. Remission via the PCL-5 is defined as a score<33
   e. Diagnosis of PTSD via the CAPS5 requires a score≥26
   f. Remission via the CAPS5 is defined as a score<26

PSSI-5 The PSSI-5 is a 34-item semi-structured symptom inventory based upon the DSM 5 criteria for the assessment of past-month PTSD. After screening for the presence of a qualifying trauma, usually the first or the worst, 20 other questions assess the following diagnostic criteria: Intrusion, avoidance, changes in mood and cognition, arousal, and hyperactivity. Items are assessed for frequency and intensity of symptoms on a 5-point scale. Symptoms are considered present with a score of 1 or more. These items result in a total symptom severity score ranging from 0 to 80. Two additional items assess for overall distress and interference with daily functioning. Two more (for a total of 24 items) assess for delayed onset and persistence of symptoms, respectively. Diagnosis depends upon the presence of 1 intrusion symptom, 1 avoidance symptom, 2 cognition and mood symptoms, and 2 arousal symptoms for a period of 1 month or greater, in addition to clinically significant distress or interference (a score of 2 or higher on either Item 21 or 22; Foa, McLean, Zang, Zhong, Rauch, Porter, . . . Kauffman, 2016). PSSI-5 showed high internal consistency (α=0.89), test-retest reliability (r.=0.87), and interrater reliability for both the total severity score (κ=0.98) and for the PTSD diagnosis (κ=0.84). It demonstrated convergent validity with CAPS for DSMS, the PTSD Diagnostic Scale for DSM 5 and PCL-S for DSM 5 (all.r=0.72). A score of 23 was identified as a cutoff score for the probable diagnosis of PTSD (Foa, et al., 2016). In accordance with PSS-I-5 application guidelines, the instrument was used to assess the identified worst, or most significant trauma (the target trauma) at intake and at the end of PTSD treatment, even if multiple traumas were treated, scores were determined based upon the initially identified target trauma.

PCL-5 The PCL-5 is a 20-item self-report inventory that evaluates past-month DSM-5 PTSD symptoms tied to patient's currently most distressing event. Items are rated from not at all (0) to extremely (4) and are summed for a total severity score. Subscale severity scores are calculated by summing items in each of the four DSM-5 PTSD symptom clusters: intrusion, avoidance, negative alterations in cognitions and mood, and alterations in arousal and reactivity. It differs from PCL for DSM IV in that it lacks separate military and specific versions. It also provides several alternate categories of DSM Criterion A not found in the earlier version (Weathers, Litz, Keane, Palmieri, Marx, & Schnurr, 2013) Wortman and Colleagues (Wortmann, Jordan, Weathers, Resick, Dondanville, Hall-Clark, . . . Litz, 2016) examined the psychometric properties of the PCL-5. It was found to display high internal consistency (α=0.95), with high convergent and discriminant validity when compared against the PSS-I for DSM IV (.r=0.94, .r=0.92, respectively). A score of 33 was reported as optimally effective in the presumptive diagnosis of any PTSD. The earliest group of trainees (n=11) used the PCL-5 to evaluate changes in patients' symptom, soon thereafter, the remaining clinicians used the PSS-I-5.

CAPS5 The Clinician-Administered PTSD Scale (CAPS) is an extensively validated and widely used structured diagnostic interview for posttraumatic stress disorder (PTSD). The CAPS was recently revised to correspond with PTSD criteria in the fifth edition of the Diagnostic and Statistical Manual of Mental Disorders (DSM-5; American Psychiatric Association, 2013). This article describes the development of the CAPS for DSM-5 (CAPS-5) and presents the results of an initial psychometric evaluation of CAPS-5 scores in 2 samples of military veterans (Ns=165 and 207). CAPS-5 diagnosis demonstrated strong interrater reliability (κ=0.78 to 1.00, depending on the scoring rule) and test-retest reliability (κ=0.83), as well as strong correspondence with a diagnosis based on the CAPS for DSM-IV (CAPS-IV; κ=0.84 when optimally calibrated). CAPS-5 total severity score demonstrated high internal consistency (α=0.88) and interrater reliability (ICC=. 91) and good test-retest reliability (ICC=0.78). It also demonstrated good convergent validity with total severity score on the CAPS-IV (r=0.83) and PTSD Checklist for DSM-5 (r=0.66) and good discriminant validity with measures of anxiety, depression, somatization, functional impairment, psychopathy, and alcohol abuse (rs=0.02 to 0.54). Overall, these results indicate that the CAPS-5 is a psychometrically sound measure of DSM-5 PTSD diagnosis and symptom severity. Importantly, the CAPS-5 strongly corresponds with the CAPS-IV, which suggests that backward compatibility with the CAPS-IV was maintained and that the CAPS-5 provides continuity in evidence-based assessment of PTSD in the transition from DSM-IV to DSM-5 criteria (Weathers, Blake, Schnurr, Kaloupek, Marx, & Keane, 2013: Abstract)
4. The Tool of Subjective Units of Distress scores (SUDs; Wolpe, 1969) is used by the clinician to probe and objectively monitor the patient's progress through treatment.
   a. SUDs are a ten-point Likert scale ranging from zero to ten that the clinician employs to quantify the relative presence or absence of fear, terror, and helplessness regarding the patient's current experience of the trauma memory.
   b. SUD scores are assessed immediately after the initial narrative, after the fast rewind, and after the best-of-all-worlds rescripting.
5. Having used the tool of diagnostic inventories, the clinician provisionally admits or rejects the patient from the procedure.
   a. Provisionally admitted patients are questioned about their trauma experience(s), their symptoms, their past treatments, and current medications.
   b. The clinician, depending upon their subjective assessment or the use of diagnostic tools, determines whether the patient is disqualified from the treatment. Observed incapacity is determinative.
      i. Unable to participate through inebriation sufficiently severe as to interfere with their ability to complete tasks as directed.
      ii. Unable to participate through mental incapacity sufficiently severe as to interfere with their ability to complete tasks as directed.
      iii. Unable to participate through the presence of Axis I disorders sufficiently intrusive as to interfere with their ability to complete tasks as directed.
      iv. Unable to participate through the presence of Axis II disorders sufficiently intrusive as to interfere with their ability to complete tasks as directed.
   c. In general, the clinician's determination, using the tool of Calibration (as trained, professional observation), is determinative.
6. The clinician uses the tools of clinical assessment to determine
   a. Completion of treatment
   b. Treatment success
7. Treatment completion occurs when the clinician determines,
   a. That the patient has completed all three stages of treatment
   b. That the patient has obtained a score<2 using the tool of Subjective Units of Distress
   c. The clinician using the tool of trained observation determines that the reported SUD score is consistent with their observation of patient physiology. To wit, the absence of the indicia of autonomic arousal as detailed in Table 1.
8. Treatment success occurs
   a. When the patient has completed treatment
   b. When the clinician, using the tool of diagnostic inventories, determines that the patient has achieved a follow-up score below clinical threshold as follows:
      i.Remission via the PSSI-5 is defined as a score<23
      ii.Remission via the PCL-5 is defined as a score<33
      iii.Remission via the CAPS5 is defined as a score<26
1. The Tool of Calibration is a tool, used by the clinician to “read” unconscious, non-verbal responses in an ongoing interpersonal interaction by pairing observable behavioral cues with corresponding internal states of consciousness or responses.
   a. The tool of Calibration entails careful monitoring of the patient for changes in that person's physiology including:
      i. posture
      ii. breathing
      iii. muscle tonus
      iv. skin color
      v. pulse
      vi. voice pitch
      vii. speech rate
      viii. other physiological responses.
   b. The Tool of calibration requires the clinician to make a baseline assessment of the patient's normal physiology in normal conversation.
   c. Those observations will include:
      i.posture
      ii.breathing
      iii.muscle tonus
      iv.skin color
      v.pulse
      vi.voice pitch
      vii.speech rate
      viii.other physiological responses
   d. Changes from baseline at specific points in the protocol alert the clinician to
      i.Rapport considerations
      ii.Memory activation
      iii.Patient cooperation with clinician directives
      iv. Increases in anxiety/fear
      v.Decreases in anxiety/fear
   e. Changes from baseline at specific points in the protocol alert the clinician to significant changes in their responses to:
      i. The trauma memory
      ii. The clinician's instructions
   f. RTM Clinician uses the tool of Calibration to establish and maintain rapport with the patient throughout the procedure to:
      i.Enhance cooperation
      ii.Support authoritative directives
      iii.Assess readiness to proceed with or end the session.
   g. RTM Clinician uses the tool of calibration at the telling of the initial narrative
      i. To quickly Identify whether the patient is only relating a sequence of events (a gist, or other semantic-level summary)
      ii.To quickly identify whether the Patient is presently re-experiencing/re-associating into an activated traumatic event.
   h. RTM Clinician uses the skill of calibration at the telling of the later narratives
      i.To quickly determine whether the patient has retained physiological reactivity to elements of the narrative
      ii.To determine whether the narrative is no longer emotionally salient.
   i. RTM clinician uses the tool of calibration to assess whether the patient has complied with or is complying with his or her directives.
   j. RTM clinician uses the tool of calibration to assess for abreaction or unwanted excessive activations of the trauma memory so that those abreactions or unwanted excessive activations can be terminated.
   k. RTM clinician uses the tool of Calibration to determine whether their implementation of a break-state tool has successfully calmed the patient.
   The Tool of Rapport
   The Narrative Parsing Tool
   The Tool of Structural Modification
   The Break State Tool
1. Whenever physiological signs of activation are observed, the clinician stops the patient's current activity and redirects them (in accordance with their training) to
   a. A different sensory system,
   b. A conversation ended earlier in the session
   c. Some special interest of the patient
   d. Imagine their telephone number written on the ceiling and reading the numbers backward.
   e. Some other complex, potentially amnestic or attention-grabbing, positive topic or sensory element.
2. Re-orient the patient and assess their emotional state.
3. The patient must be calm.
4. This requires ongoing assessment by the clinician using the Tool of Calibration

### The Activation Parsing Tool

1. Human autobiographical memories exist at multiple levels of integration. There is evidence that any emotional memory may exist simultaneously at different levels of abstraction that all are more or less available to conscious access. (Greenberg, & Verfaellie, 2010; Hassabis & Maguire, 2009; Renoult et al., 2021; Xue, 2022)
2. These may include
   a. Gists (abstract representations of the essential aspects and/or theme of an event! Addis, 2020 p. 235)
   b. Schemas (high-level knowledge structures that organize lower-level forms of memory I Addis, 2020 p. 235)
   c. semantic categories (categories are networks of associated concepts that in turn, are a set of defining features! Addis, 2020 p. 235)
   d. episodic memories at various levels of sensory re-experiencing
   e. and Moreover, repeated episodic recall gives rise to more abstract gists and schemas, while gists and schemas scaffold episodic memory (Addis, 2020; Greenberg, & Verfaellie, 2010; Moscovitch, 2008; Moscovitch and Gilboa, 2021; Renoult, & Rugg, 2020; Renoult, Irish, Moscovitch, & Rugg, 2019; Strange, & Takarangi, 2015; Xue, 2022).
3. Hotspots are specific emotional events or scenes that occur within a narrative and are characterized by excessive salience and increased emotional impact (Clark & MacKay, 2015).
   a. Hotspots are the elements most likely to dissociate from the larger narrative.
      i.Hotspots appear in the sequence established by the narrative or gist.
      ii.Each hotspot may be dealt with as.
         1. An experience of discomfort in any part of the black-and-white movie
         2. An element of the rewind that is difficult to undo.
         3. A particularly salient element in the rewind or any narrative.
      iii.Hotspots are resolved when.
         1. Adjustments of black and white movies increase patient comfort and allow the intervention to proceed.
         2. The rewind element is separated into sensory dimensions, which are undone one at a time.
         3. Any narrative element, or another part of the process, may become the focus of narrowing bookmarks, undoing, or more movies with adjustments.
   d. In light of these considerations, there are several kinds of Observable activation styles that may be used by the clinician to parse the expected activation style using the tool of Calibration.
   e. These may include:
      a. Activation one—Simple declarative access to episodic experience with UCS as a cue
         i. The RTM Narrative is a typical UCS.
         ii. The entire event was activated via the presentation of the UCS.
         iii.Stimulus onset (the narrative's beginning) usually produces an observable response.
         iv. The observable response is the only reliable index of memory activation and must be terminated immediately.
         v.Expect fast resolution.
         vi. In cases of simple PTSD and newer cases, patients may labilize an entire event at once.
         vii. The activation may be so immediate and dramatic that the procedure must be followed content-free. These cases can expect fast resolution with one pass through the protocol necessarily involving multiple experiences of the black-and-white movie with modifications, several rewinds, and numerous practices of the best-of-all-worlds scenario.
   b. Activation two—link to simple declarative UCS via CS/trigger.
      i.Activation of UCS/declarative memory via single CS/reminder.
      ii.All triggers activate the same event via a single hotspot.
      iii. The CS activates the memory via simple association.
      iv. The hotspot is the entire trauma.
      v.Activation may be slightly slower than in type 1.
      vi.Stimulus onset (the narrative's beginning) usually produces an observable response.
      vii. The observable response is the only reliable index of memory activation and must be terminated immediately.
      viii. In some cases, the activation is so fast and dramatic that the procedure must be followed content-free.
      ix.Expect fast resolution.
   c. Activation 3—Multiple CS/triggers/hotspots scaffolded together as a single event.
      I.Flat affect for scaffold (schema, gist, or summary) with minimal affect for one or more hotspot(s).
      II. The memory of the trauma has been distributed across the cortex with a cold narrative linking the emotionally more salient narrative elements.
      III.As each memory element or potential hotspot is accessed, it may be revivified by manipulating sensory elements, as in the scene adjustments during the black-and-white movies. Scene adjustments are not only the means whereby the salience of the memory and its components are adjusted but also serve as a means for re-accessing the original sensory dimensions of the target memory.
      IV.As the patient and clinician adjust the salience-related dimensions of the trauma movie, there is an essential, typically unconscious, recall of the original sensory data and its subsequent modification under the clinician's instructions.
      V.Hotspots appear in the sequence established by the Narrative or gist.
   d. Activation 4—Gist with a highly practiced presentation and minimal involuntary responding.
      I.Flat affect for scaffold with minimal affect for one or more hotspot(s).
      II. The memory of the trauma has been distributed across the cortex with a cold narrative linking the potentially more emotionally salient narrative elements.
      III.Activate available hotspots.
      IV.Be sensitive to hot spots in the movies where the patient suddenly requires new adjustments, e.g.., when:
         1. the patient complains of his own discomfort
         2. displays signs of autonomic arousal
         3. reports that their avatar in the theater is uncomfortable or restless.
         4. Occasions when, after a narrative, the SUDS value suddenly jumps. Review the current activity level and determine what adjustments to the movie or rewind will satisfy the current need.
   i. In most such cases, the hotspots will be resolved individually as the patient proceeds through the protocol
   j. Memory elements may arise as hotspots that can be dealt with through the protocol's recursive nature.
   k. Whereas hotspots are treated in the repeated black-and-white movies and rewinds, using structural adjustments, the larger frames, the gists, and schemas are integrated through the retelling of the narrative and the Best-of-all-worlds scenario.
1. If no Hotspot or autonomic response can be ascertained
   a. Switch to another trauma (if available)
   b. Ask the patient what they think a good place to start might be (i.e. If they were to respond, which part of the narrative would they be more likely to respond to?).
   c. In discussion with the Patient, move the bookmarks closer to the actual temporal limits of the targeted hotspot.
      i.Each memory element or potential hotspot is accessed, it may be revivified by manipulating sensory elements, as in the scene adjustments in the black-and-white movies.
      i. The scene adjustments are not only the means whereby the salience of the memory and its components are adjusted but also a means for re-accessing the original sensory dimensions of the target memory.
      ii.As the patient and clinician adjust the salience-related dimensions of the trauma movie, there is an essential, typically unconscious, emergence of the original sensory data.
   e. Activation 5—Highly emotional narrative with minimal progress. Progress may occur as the patient considers that things might have gone another way.
      i. The patient is generally unable to review trauma memory.
      ii. In the Black and White Movie scenario there is some minor progress but the patient still refuses to face the target memory, even though the exposure will be limited.
      iii.After a few sessions, the patient begins to wonder if they could have done something different or gone another way.
      iv.Move to the Best-of-all-worlds restructuring.
      v.Build a strong new representation and practice 8 to 10 times.
      vi.Complete SUDs
      vii.Proceed through the protocol normally.
      viii.Be sensitive to hot spots in the movies where the patient suddenly requires new adjustments, such as when:
         A. the patient complains of his own discomfort,
         B. displays signs of autonomic arousal,
         C. reports that their avatar in the theater is uncomfortable or restless. after a narrative, the SUDS value suddenly jumps. Review the current activity level and determine what adjustments to the movie or rewind will satisfy the current need.

In most such cases, the hotspots will be resolved individually as the patient proceeds through the protocol. Memory elements may arise as hotspots that can be dealt with through the protocol's recursive nature.

Whereas the hotspots are treated in the repeated black-and-white movies and rewinds, using structural adjustments, the larger frames, the gists, and schemas are integrated through the retelling of the narrative and the Best-of-all-worlds scenario.

As each memory element or potential hotspot is accessed, it may be revivified by manipulating sensory elements, as in the scene adjustments in the black-and-white movies. The scene adjustments are not only the means whereby the salience of the memory and its components are adjusted but also a means for re-accessing the original sensory dimensions of the target memory. As the patient and clinician adjust the salience-related dimensions of the trauma movie, there is an essential, typically unconscious, emergence of the original sensory data.

RTM is a sophisticated neurological intervention for the successful resolution of PTSD and/or its symptoms. It is not a therapy. That is, it is not a structured intervention aimed at the mollification of symptoms. RTM is a structured intervention that uses the modification of the structure of a targeted imaginal memory structure within a reconsolidative memory regime to change the salience of the patient's experience. By performing the change in the context of reconsolidation, the source memory is restructured in a relatively permanent fashion. By restructuring the memory in a manner consistent with salience markers in the superior colliculus, we are able to lessen their perceived importance. When the importance or salience of the memory is decreased, it can no longer be remembered as a present-time emergent threat.

RTM takes advantage of specific biological and neurological mechanisms to directly impact the targeted memory images. These cannot be done alone as they require the external monitoring and guidance of a trained professional.

The presence of the professional is required to 1. Monitor the patient's physiology for the activation of the target memory. 2. Terminate the patient's response as quickly as possible to prevent abreaction and/or extinction using “break State” techniques. 3. Re-orient the patient to the present and assess for continuing distress. 4. Monitor progress and assess the need to repeat certain segments of the protocol.

Monitor the patient's physiology for the activation of the target memory. Unlike standardized breeds in other models whose (rats, mice, macaques) memory activation time can be simply calculated as a specific time (e.g., 30 sec, 3 min), humans display idiosyncratic activation times linked to how abstracted, as gists, summaries, schemas, etc., the target memory has become. Abstraction adds processing time to get to the emotional level required for RTM. As a result, humans must be monitored for signs of activation. Signs of activation may include sweating, tensing, reddening, changes in breathing, voice tone and rate, etc. These observations require training and practice. GSR, HRV, Startle, etc., have varying lag times in humans and often miss the critical onset of memory labilization. Verbal reports are often unreliable.

The method and tools of the invention include: A method for remission of PTSD in a diagnosed patient by a clinician using a calibration tool comprising sensors for measurable indicia of a mental state consisting of a group of indicia including breath rate, pupil dilation, eye movement, posture, speech rate, change in speech rate, change in speech volume, change in skin tone, and heart rate wherein these factors are calibrated to an individual patient and at least three factors are monitored to measure a state of activation of an index memory in said patient wherein a quiescent state of non-activation is compared against a state of dissociation from the target memory, an activated memory about to be prevented from becoming fully conscious, and an activated memory that has become fully conscious and must be terminated.

A calibration tool for assessing mental states of a person including
1. regular state—functional capable of following directions, (non-agitated or otherwise anxious)
2. 2a. Memory-activated state memory is activated and conscious elaboration prevented or terminated quickly
   2b. Memory activated with full emotional involvement (Needs break state and significant calming)
3. Abstracted state—memory is activated (2a) the patient is calm enough to follow directions
4. 4a. Break state stress level1 (Memory is activated as evidenced by signs of ANS activation without full conscious involvement break state terminates conscious activation)
   4b. Break state stress level2 Memory is activated 2b but strong conscious abreaction is present, and patient requires intense calming and reorientation.
   4c. Break State stress level3 activation during other programmatic elements other than the narration which ay be managed as in 4a &4b.

Where the tool comprising means for monitoring of breath rate, pupil dilation, eye movement, posture, speech rate, change in speech rate, change in speech volume, change in skin tone, and heart rate, facial expression, galvanic skin reaction and posture, wherein at least three of said factors are chosen. Each of these factors has known sensors that measure in near real time. These sensors include: many commercially available sensors that offer reliable ranges that can be used to assess the mental state being observed.

An interface is provided so that a clinician can choose the factors that are most pertinent to monitor. This can be a break state tool for terminating the activation of a traumatic memory before becoming fully conscious, or after its full conscious expression comprising a calibration tool comprising sensors for measurable indicia of a mental state consisting a group of indicia including breath rate, pupil dilation, eye movement, posture, speech rate, change in speech rate, change in speech volume, change in skin tone, and heart rate wherein these factors are calibrated to an individual patient and at least three factors are monitored to measure a state of activation of an index memory in said patient wherein a quiescent state of non-activation is compared against a state of dissociation from the target memory, an activated memory about to be prevented from becoming fully conscious, and an activated memory that has become fully conscious and must be terminated; wherein the clinician provides pretested directions to the patient to calm the patient and reorient them to the present clinical context.

A subjective units of distress score tool, (SU DS) for the objective assessment of subjective response. A Likert scale, from one to 10 indicating the severity of subjective fear, terror, or helplessness, used to mark patient progress through the protocol that has been physiologically calibrated using a tool comprising using a calibration tool comprising sensors for measurable indicia of a mental state consisting of a group of indicia including breath rate, pupil dilation, eye movement, posture, speech rate, change in speech rate, change in speech volume, change in skin tone, and heart rate wherein these factors are calibrated to an individual patient and at least three factors are monitored. to measure a state of activation of an index memory in said patient wherein a quiescent state of non-activation is compared against a state of dissociation from the target memory, an activated memory about to be prevented from becoming fully conscious, and an activated memory that has become fully conscious and must be terminated.

A method of claim one wherein the calibration tool indicates that the target memory has been activated and its conscious expression terminated so that the patient is calm and able to follow instructions; The clinician instructs the patient to name the memory with a neutral descriptive name as if viewing the memory from a distance.

A method wherein the calibration tool indicates that the target memory has been activated and its conscious expression terminated, so that the patient is calm and able to follow instructions; The clinician instructs the patient to identify times just before the target event occurred and just after the event had ended, bookending the event and providing named stimuli for later use.

A method where the calibration tool indicates that the target memory has been activated and its conscious expression terminated so that the patient is calm and able to follow instructions; The clinician instructs the patient to imagine a movie theater and creates a level of abstraction that manages the patient is capable of following instructions in a timely manner. This method can be accomplished by clinician instructing the patient to describe a theater, find a seat in the theater, associate fully into the theatre context, and notice on the screen a black and white still image of the first bookend before the traumatic event. The method continues with instructions to the patient, having oriented to the imaginal theater, is instructed to imagine leaving his body in the theatre, floating up into the projection booth, and associating into that context. This step provides an additional level of abstraction between the patient and possible abreaction to the memory.

The clinician instructs the patient, having associated into the projection booth is then instructed that he is not to watch the movie to be played in the theater, but he is only to watch the self, seated down in the theatre as they watch the movie. The patient is then instructed he is in charge of running the projector and will run a fast, black and white movie of the trauma event in 45 seconds or less, for the self, seated in the theater. And that he is to stop the movie if he observes, calibrates, or otherwise understands that the self in the theatre is showing signs of ANS activation. This step in monitored by the calibration tool to insure ANS activation is averted by use of the termination tool using any three of the indicia from method one, calibrates that the patient is showing signs of ANS activation, they will break state and calm the patient The patient having associated into the projection booth is instructed by the clinician that in the case that he or the self in the theatre are activated by the running of the movie, the patient is to ensure that he is not watching the movie, but only watching the self in the theater and that he is remaining in the projection booth and not associating into the self in the theatre.

Having reached the previous step wherein the patient, having associated into the projection booth, is instructed by the clinician that as projectionist he can modify the structure of the movie so as to make its viewing more comfortable for the self in the theatre. Those modifications may include the distance of the screen, size of the screen, size of the movie on the screen, aspect ratio, lateral and horizontal leaning of the screen, blurring of the screen image, viewing the movie as stick figures, viewing the movie as a cartoon, floating above the theatre and viewing the projectionist and self in the theatre from above—singly or in combination and with each showing of the movie with calibrated activation, more structural changes can be made or otherwise modified or eliminated.

The next steps in the abstraction process in a mental condition that ensures the patient can follow directions and avoid abreaction allow modification of the trauma index memory structure. More specifically, the patient, having associated into the projection booth is instructed by the clinician to run the movie multiple times punctuated by collaborative discussions with the clinician of how to modify the structure of the movie until, and running the movie with those modifications, by asking and using the calibration tool, the clinician determines that the patient is no longer reactive, the clinician instructs the patient to repeat the movie 8 to 10 times with the scene just after the event remaining on the screen.

A test for the absence of ANS activation, showing no stress, the clinician instructs the patient to return to his body in the theatre and to determine whether he is comfortable in that body. The patient returns to the body in the theater and assesses for comfort; the clinician, uses the calibration tool to confirm the activation state of the patient and determines whether or not to use the break state tool (Method new 2) and instructs the patient to return to the projection booth and continues from the method of claim 10 forward. Then the clinician assesses the patient's activation state and determines if the patient remains calm, the clinician now instructs the patient to stand up, walk to the front of the theatre, and step into the scene in the movie depicting the second bookmark, now projected in black and white on the screen. Continued monitoring with the calibration tool the clinician instructs the patient has stepped into the scene of the second bookmark, the clinician instructs the patient to turn on the color and sound, to make the scene 3 dimensional and to run the experience, the entire multi-sensory movie, backward, from within their own body from end to beginning in three seconds or fewer.

The ensuing steps follow: 1. the clinician assesses the patient's response using the calibration tool to determine the presence or absence of ANS responsivity after the first running of the reversed movie; 2. The clinician if he assesses stress as indicated by three or more of the indicia of the calibration tool, that the patient has increased reactivity, the clinician, using the break state, calms and reorients the patient before continuing with several more rewinds.

If the clinician assesses the patient's response to the movie reversal using three or more of the indicia of claim one, finding no ANS reactivity, instructs the patient to repeat the reversed movie five to seven more times. After completing 5 to 7 repetitions of the reversed movie the clinician assesses the patient for three or more of the indicia of ANS activation and if assessed that the patient is activated, the clinician uses the break state tool and having determined that the patient is calm and oriented to the present determines whether to repeat the reversed movie five to seven more times If having determined that the patient is no longer reactive, the clinician instructs the patient to relate the trauma narrative as he uses the calibration tool to assess the patients activation state and can avert a panic reaction by using the break state tool to disassociate the patient from an abreaction to the memory. Having determined by three or more of the indicia being monitored by the calibration tool, that the patient is still activated on retelling the trauma narrative, the clinician uses the break state tool which calms and reorients the patient.

The clinician then chooses whether having calmed and reoriented the patient whether to refer the patient to the method of having the patient change the level of abstraction from the memory or instructs the patient to repeat the reversed movie five to seven more times.

Testing can also include the clinician instructing the patient after having completed five to seven more reversed movies in, and observing no further activation with via three or more of the indicia of stress the patient is again instructed to repeat the trauma narrative, as the clinician assesses for ANS activation using the calibration tool. Finding no ANS activation during the narrative, the clinician instructs the patient to use the SUDS tool) to provide an assessment of their current status. If the SUD score is 3 or higher, and the assessment accords with calibration tool he refers the patient to the further work in either the abstraction protocol.

If the SUD score is 3 or lower and accords with the clinician's assessment using the method of claim 4, the clinician now instructs the patient to create a new version of the trauma movie beginning at the first bookend, before the traumatic event occurred, and may, from there. change the rest of the story so as to create a best-of-all-worlds version of the original movie that does not activate the patient and may completely depart from the original narrative. With an acceptable revised narrative the clinician now instructs the patient to run this new version as a dissociated movie. The clinician now instructs the patient to run the revised movie five to seven times with a pause between each, while the clinician keeps count.

When the this abstraction protocol has been completed the patient is instructed to complete a SUDs assessment achieving a score of two or greater, and this score according with the clinician's assessment using the calibration tool the patient may be instructed to review the revised movie and discover how to improve it; The clinician instructs the patient to return to the abstraction 0 and to proceed from there.
1. The method of claim 35 having failed to produce a SUDS of 0 or 1, the clinician determines whether to refer the patient to the method of claim 28.
2. The method of claim 34 having produced a SUDS of one or zero, and in accord with the clinician's assessment using the calibration tool of claim 4, the treatment is deemed complete.

The calibration tool allows both measurable factors to be measured and observable behaviors to be monitored and quantified. Factors such as respiration rate, heart rate, blood pressure, speech rate, pupil dilation, etc. can be automatically measured, and changes in the factors can be noted. Factors such as speech cadence, tone, attitude, facial expressions, and posture can be observed, and interpreted. As importantly observable factors which are less directly interpreted, can be coordinated with the readily measured factors, and interpreted.

The calibration tool can be made from a collection of tools ad sensors that collect data using sensors such as heartbeat monitors, skin contact electrocardiograms, blood pressure monitors, non-contact thermometers, skin contact galvanometers, oxygen sensors. Each of these feed the desired data in to real time data recorders where various limits are used as indicators of mental state; disassociated, memory activation, heightened stress and panic reaction.

At least four mental states that can be measured with tools and sensors relate to a specify process/protocol. The four mental states are:
a. Regular state—functional capable of following directions, (non-agitated or otherwise anxious).
b.1. Memory-activated state memory is activated and conscious elaboration prevented or terminated quickly
b.2. Memory activated with full emotional involvement (Needs break state and significant calming).
c. Abstracted state—memory is activated (2a) the patient is calm enough to follow directions.
D.a. Break state stress level1 (Memory is activated as evidenced by signs of ANS activation without full conscious involvement break state terminates conscious activation).
D.b. Break state stress level2 Memory is activated 2b b.2 ut strong conscious abreaction is present, and patient requires intense calming and reorientation.
D.c. Break State stress level3 activation during other programmatic elements other than the narration which may be managed as in Da & D.b.

Activation is time-sensitive, and reconsolidation will fail if the target memory image is allowed to persist in violation of the time constraint. The result will be either retraumatization or an extinction regime.

Terminate the patient's response. Once the physiological signs of activation are observed, the clinician stops the patient's narrative and redirects them (in accordance with their training) to a). a different sensory system, b). a conversation ended earlier in the session, c). some special interest of the patient, or d). Some other potentially amnestic or attention-grabbing, positive topic or sensory element.

The trauma story is irrelevant to the intervention. This is a neurological intervention by a trained professional who guides the patient's efforts in order to ensure that a). The memory is activated; and b). Conscious focus on the memory is terminated as quickly as possible after activation. The patient relates the narrative for the sole purpose of activating the target memory. Later (during the active intervention), the activated memory will be susceptible to modification.

The nature of PTSD is such that activation of the memory(ies) at the heart of the malady usually results in consequences that may last for hours or days as intrusive thoughts, altered mood, flashbacks, nightmares, depression, and suicidal ideation. These responses occur outside of the patient's control and require the monitoring and management of a trained professional. In severe cases, lack of management here may result in significant abreaction and reportable adverse incidents, with the possible need for psychiatric intervention. It would be unethical to allow patients to attempt RTM without a trained guide.

The intervention hangs upon the successful activation and fast termination of the patient's recall of the target memory. Although the protocol offers many opportunities for activation and re-activation of the target memory(ies), the early, expert management of memory activation is the stone of stumbling that afflicts many reconsolidation studies.

1. Re-orient the patient and assess their emotional state. Before the intervention may proceed, the patient must be calm. This requires ongoing assessment by the clinician and their willingness to adjust programming based upon their observations of the patient's state. The clinician must have sufficient expertise to be able to interpret the patient's physiology and to ignore (when necessary) verbal assessments by the patient in favor of their own expert observations of the patient's physiology.

2. Monitor progress and assess the need to repeat. The clinician must remain sensitive to the patient's changing physiology, and their verbal and non-verbal responses. They must be ready to quickly end the expression of strong negative emotion so that each is labilized so that it can be adjusted with other elements of the larger situational memory. Newly recalled traumas will often arise after the initial target memory has been dealt with so the clinician must be ready to deal with each or any that arise. In cases of complex PTSD these newly emergent traumas will typically be overwhelming and require the care of a trained clinician for breaking state, calming the patient, and reorienting them to the present.

Calibration takes an array of sensors or monitors a selection of the factors from the group consisting of breath rate, pupil dilation, eye movement, posture, speech rate, change in speech rate, change in speech volume, change in skin tone, and heart rate, and monitors them especially when the index trauma memory is being addressed, or safe state memories are explored and identified. So a subset of the inputs are selected, the physiological measurements can be measured and monitored by measuring devices such as heartbeat monitor, thermometer monitors, galvanic skin sensors, respiration rate monitors, blood oxygen sensors, blood pressure monitors. All of these devices are commercially available and when selected are linked either by cable, or wirelessly to a computerized wireless receiver that displays these factors and compares them to determined limits for each of the factors that have been calibrated to indicate stress.

Autonomous factors such as posture, speech rate, speech volume, facial expression and can be monitored by video recognition and determined to above or below set standards calibrated for the patient.

These two factors and direct observations by the clinician of expressive behavior, rather than the expression content, cue the clinician as to the patient's mental states including memory activation, memory labilization, stress calibrated to a standard of subjective distress, severe stress, and relative disassociation. With the calibration monitor and calibration, the clinician reads the actual mental state, in regards to its level of distress directly, identifies when specific memories are activated and labile, and enables the clinician to conduct specific tasks safely and effectively.

Upon determination of labialization of the index trauma memory the clinician can start conducted abstraction tools to further reduce the salience of the memory labilize the memory while maintaining a low level of distress. The Theater protocol of discussion allows the patient to safely experience the trauma memory and activate safe state memories associated in time with the trauma memory and alter the structure of the memory.

The clinician can also monitor the patient's stress level and instruct the patient when they are still capable of following directions to focus on specific safe ideas, subjects, or memories that have been pre-tested to be unstressful subjects that have been put into an inventory of memories that do no create stressful memories. The inventory of break state directions have also been tested by the calibration tool as the tool was built.

The correlation between the measurable factors and clinician observation is another benefit of the calibrations tool.

What is claimed is:

1. A calibration tool for determining the mental stress level of a patient wherein factors from the group consisting essentially of breath rate, pupil dilation, eye movement, posture, speech rate, change in speech rate, change in speech volume, change in skin tone, and heart rate, wherein at least three of said factors are chosen by a clinician to monitor wherein the three factors are chosen that represent objective characteristics of a patients anxiety state ranging from traumatic memory activation to traumatic memory reaction, but where a state less than traumatic memory reaction is used to provide a signal to said clinician to provide directions to said patient to disassociate from said traumatic memory.

2. A method for remission of PTSD in a diagnosed patient by a clinician using a calibration tool comprising: sensors for measurable indicia of mental state consisting of the group of indicia of breath rate, pupil dilation, eye movement, posture, speech rate, change in speech rate, change in speech volume, change in skin tone, and heart rate, wherein these factors are calibrated to an individual patient and at least three factors are monitored to measure an activation state of an index memory in said patient wherein the calibration tool is calibrated to test for activation of the memory and a state of dissociation from the memory as indicated by the patient shown by their ability to follow directions from said clinician.

3. The method of claim 2 wherein when the calibration tool indicates activation of said traumatic memory the clinician instructs the patient to imagine a theater where the patient imagines seeing themselves view a movie of the traumatic event, but the patient only views themselves viewing said movie, the clinician then uses the calibration tool to insure the patient has activated said traumatic memory while they are also not at risk of re-traumatizing themselves.

4. A break state tool for disassociating a patient from a traumatic memory comprising; a library of interests which a patient has demonstrated performance in responding to the tool when instructed. The tested break state cues are tried out, recorded and assessed for their effectiveness and are used when a calibration tool exceeds a measured indication of patient anxiety.

5. A test for determining PTSD remission where the calibration tool of claim 1 shows no stress in the patient when the patient discusses the index memory.

6. The method of claim 2 further comprising the steps of having a patient guided by a clinician apply the syntax of reconsolidation blockade to the treatment of PTSDhaving said clinician identify acceptable patients; having said clinician identify an individual acceptable patient and wherein said patient has experienced multiple traumas separating multiple traumas into discrete events; framing the intervention and creating rapport; eliciting and terminating the trauma memory without retraumatizing the patient; identifying autonomic arousal by use of the calibration tool described in claim 3; restructuring the emotional experience of the traumatic event; testing the efficacy of the intervention; modifying the subjective structure of representations within the trauma memory using the theater protocol.

7. The method of claim 2 further comprising the steps of a) establishing rapport with the patient, and using said calibration tool to measure and record factors chosen from measurable factors and behaviors of said patient chosen from the group consisting of pulling in said factors; wherein said tool measures memory activation level of said trauma index, and the patient's measurable factors and behavior leading to pre-panic anxiety state b) discovering the index trauma of said patient, c) identifying and separating discrete traumas of said patient, d) eliciting from said patient and terminating a brief re-experience of the index trauma, e) guiding the patient through the treatment, preventing retraumatizations and abreactions by said patient, f) determining which modifications to the intervention are appropriate, and g) testing for completion of treatment.

8. The method of claim 7 wherein the clinician applies inclusion criteria comprising a) a determination of whether the index trauma memory is rooted in the personal experience of trauma threatening death or injury to one's self or others, b) a determination of whether the patient exhibits a phobic, instantaneous conditioned response to: the narrative of, an element of that narrative, or a stimulus related to a specific traumatic event, c) a determination of whether those responses are typically expressed as an intense, suddenly arising experience of the trauma symptoms usually experienced as flashbacks or panic reactions, and d) a determination of whether those flashbacks and or nightmares are experienced at least once per month.

9. The method of claim 8 wherein the clinician applies exclusion criteria comprising a) a determination of whether the patient's presenting symptoms are a response to the meanings of the event in the patient's larger life, b) a determination of whether the symptoms are primarily a response to the impact of traumatic events on the patient's sense of self-worth, c) a determination of whether the symptoms are centered on dissociative and avoidant symptoms and not centered on flashbacks, nightmares or other intrusive symptoms.

10. The method of claim 9 wherein the clinician determines an index trauma using probes comprising: a) asking the patient, what is the central or most important problem, b) asking the patient which event most closely resembles the dream contents of nightmares or the delusional aspects of flashbacks.

11. The method of claim 10 whereby the clinician determines the limits of the index trauma by means comprising discovering with the patient a distinct beginning and end point for each traumatic event.

12. The method of claim 10 wherein the beginning points and end points of said traumatic event are points in time before the beginning of and after the end of said traumatic event.

13. The method of claim 14 wherein the clinician elicits the index trauma using probes comprising a) asking simple questions, b) inviting the patient to imagine the experience the trauma event, c) Introducing specific visual or auditory stimuli known to involuntarily evoke the experience.

14. The method of claim 2 wherein the clinician terminates the patient's experience of the index trauma.

15. The method of claim 14 wherein the clinician terminates an experience of the index trauma using various means individually and in combination comprising a) distracting the patient so as to return them to a neutral or resourceful state by asking the patient about their favorite music, favorite foods, where they grew up, or any other innocuous topic; b) Looking the patient in the eye and calling their name while instructing them to be present; c) picking up a conversation that ended before initiation of treatment as if treatment had never happened; d) Moving the patient to a different physical perspective; e) Orienting the patient to a different sensory system; f) Having the patient become aware of their feet on the floor and their presence in the room.

16. The method of claim 2 wherein the clinician prevents abreactions and retraumatizations through following an abstraction protocol comprising the steps of a) observing the patient for signs of autonomic arousal; b) noting the speed with which autonomic arousal is occurring; c) directing the patient to follow directions to reach a breaking state and ending the arousal.

17. The method of claim 16 wherein the behavioral interventions employed by the clinician comprise directions to the patient to imagine variants of the sensory elements that structure the intervention wherein the variants of the sensory elements related to the presentation of the. imagined movie screen that comprise: a) a movie screen; b) a wall; c) the inner eyelids; d) other imagined projection surface.

18. The method of claim 17 wherein the direction variants of the sensory elements comprise changing the distance between the observer and the imagined projection surface.

19. The method of claim 18 wherein the direction variants of the sensory elements comprise changing the angle of view or presentment of the imagined projection surface.

20. The method of claim 19 wherein the direction variants of the sensory elements comprise changing the position of the patient in the imaginal theater so as to change their angle of view with regard to the imagined projection surface.

21. The method of claim 20 wherein the direction variants of the sensory elements comprise viewing only parts of the imaginal trauma movie, one at a time.

22. The method of claim 17 wherein the direction variants of the sensory elements comprise viewing the top half or third of the trauma movie all of the way through, followed by the middle half (or third) of the imaginal trauma movie all of the way through, etc., one at a time.

23. A break state tool for terminating the activation of a traumatic memory before becoming fully conscious, or after its full conscious expression comprising a calibration tool comprising sensors for measurable indicia of a mental state consisting of a group of indicia including breath rate, pupil dilation, eye movement, posture, speech rate, change in speech rate, change in speech volume, change in skin tone, and heart rate wherein these factors are calibrated to an individual patient and at least three factors are monitored to measure a state of activation of an index memory in said patient wherein a quiescent state of non-activation is compared against a state of dissociation from the target memory, an activated memory about to be prevented from becoming fully conscious, and an activated memory that has become fully conscious and must be terminated; wherein the clinician provides pretested directions to the patient to calm the patient and reorient them to the present clinical context.

24. A subjective units of distress score tool, (SUDS) for the objective assessment of subjective response. A Likert scale, from one to 10 indicating the severity of subjective fear, terror, or helplessness, used to mark patient progress through the protocol that has been physiologically calibrated using a tool comprising using a calibration tool comprising sensors for measurable indicia of a mental state consisting of a group of indicia including breath rate, pupil dilation, eye movement, posture, speech rate, change in speech rate, change in speech volume, change in skin tone, and heart rate wherein these factors are calibrated to an individual patient and at least three factors are monitored to measure a state of activation of an index memory in said patient wherein a quiescent state of non-activation is compared against a state of dissociation from the target memory, an activated memory about to be prevented from becoming fully conscious, and an activated memory that has become fully conscious and must be terminated.
